(19) United States
(12) Patent Application Publication
(10) Pub. No.: US 2014/0148464 A1
(43) Pub. Date: May 29, 2014

(54) SALT FORMS OF FATTY ACIDS FOR THE TREATMENT OF DRY EYE

(71) Applicant: Thetis Pharmaceuticals LLC, Southport, CT (US)

(72) Inventors: Banavara L. Mylari, Waterford, CT (US); Frank C. Sciavolino, Waterford, CT (US); Gary Mathias, Southport, CT (US)

(73) Assignee: Thetis Pharmaceuticals LLC, Southport, CT (US)

(21) Appl. No.: 14/092,005

(22) Filed: Nov. 27, 2013

Related U.S. Application Data
(60) Provisional application No. 61/730,173, filed on Nov. 27, 2012.

(51) Int. Cl. A61K 31/495; A61K 31/202

(52) U.S. Cl. CPC A61K 31/495; A61K 31/202
USPC 514/252.12; 514/560

(57) ABSTRACT

Provided herein are compositions for the treatment of dry eye, comprising a pharmaceutically acceptable carrier and a pharmaceutically effective amount of a fatty acid salt, and methods of use thereof.

### RELATED APPLICATION

This application claims priority to U.S. Provisional Application No. 61/730,173, filed Nov. 27, 2012. The content of this application is incorporated herein by reference in its entirety.

### BACKGROUND OF THE INVENTION

Dry eye or dry eye disease (also known as keratoconjunctivitis sicca) is a prevalent health problem affecting millions of Americans each year, particularly older populations and women. Thus, as the population ages, the burden of dry eye associated morbidity will continue to grow. In mild cases of dry eye, a patient may experience burning, a feeling of dryness, and persistent irritation, while in severe cases of dry eye, vision may be substantially impaired. These symptoms affect performance of daily activities and can lead to a decrease in the quality of life and consequently increase the risk of experiencing symptoms of anxiety and depression.

Accordingly, there remains a need for treating dry eye.

### SUMMARY OF THE INVENTION

Provided herein are compositions for the treatment of dry eye, comprising a pharmaceutically acceptable carrier and a pharmaceutically effective amount of a fatty acid salt, and methods of use thereof.

In one aspect of the invention, a composition for the treatment of dry eye is provided, comprising a pharmaceutically acceptable carrier and a pharmaceutically effective amount of a fatty acid salt of Formula I:

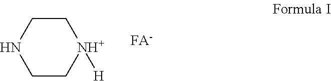

a fatty acid salt of Formula II:

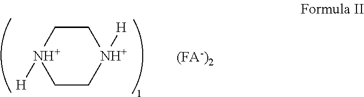

a fatty acid salt of Formula III:

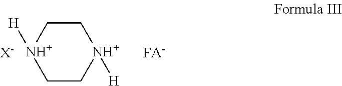

a fatty acid salt of Formula IV:

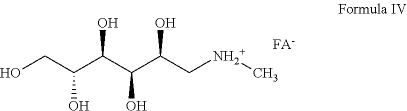

a fatty acid salt of Formula V:

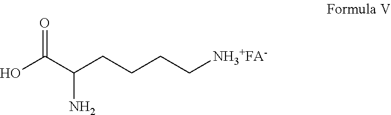

a fatty acid salt of Formula VI:

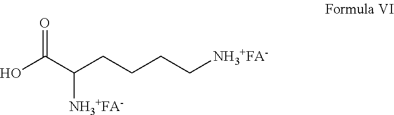

a fatty acid salt of Formula VII:

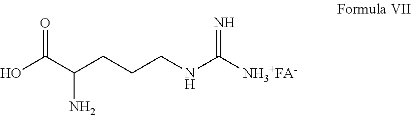

or a fatty acid salt of Formula VIII:

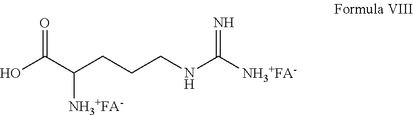

wherein FA⁻ is an anion of an omega-3 or omega-6 polyunsaturated fatty acid, and X⁻ is an anion of a pharmaceutically acceptable acid compound, or a combination thereof.

According to one embodiment, the fatty acid salt is a compound of Formula I, Formula II, or Formula III.

According to another embodiment, FA⁻ is an anion of an omega-3 fatty acid, and the omega-3 fatty acid is selected from the group consisting of alpha-linolenic acid, stearidonic acid, eicosapentaenoic acid, and docosahexaenoic acid.

In a further embodiment, FA⁻ is an anion of an omega-3 fatty acid, and the omega-3 fatty acid is eicosapentaenoic acid or docosahexaenoic acid.

According to yet another embodiment, FA⁻ is an anion of an omega-6 fatty acid, and the omega-6 fatty acid is selected from the group consisting of linoleic acid, gammalinolenic acid, dihomogammalinoleic acid, and arachidonic acid.

In another embodiment, the composition is formulated for topical administration to the eye of the subject in need thereof.

In still another embodiment, the composition further comprises one or more of glycerin, hypromellose, propylene glycol or polyethylene glycol.

According to one embodiment, the composition further comprises one or more of polysorbate 80, carbomer copolymer type A, purified water, sodium hydroxide, ascorbic acid, benzalkonium chloride, boric acid, dextrose, disodium phosphate, glycine, magnesium chloride, potassium chloride, sodium borate, sodium chloride, sodium citrate, sodium lactate, edetate disodium, hydrochloric acid, sodium hydroxide, aminomethylpropanol, hydroxypropyl guar, polyquaternium-1, or sorbitol.

According to another embodiment, a method is provided, comprised of administering a composition for the treatment of dry eye in a subject in need thereof.

Another embodiment provides a method wherein dry eye is selected from the group consisting of alacrima, xerophthalmia, Sjogren's syndrome associated dry eye, non-Sjogren's syndrome associated dry eye, dry keratoconjunctivitis, ocular pemphigoid, dry eye after ophthalmic operation, and dry eye accompanied with conjunctivitis.

Still another embodiment provides a method wherein dry eye is aqueous tear-deficient dry eye or evaporative dry eye.

According to another embodiment, a method is provided for increasing tear production in a subject comprising administering to the subject a composition for the treatment of dry eye.

In yet another embodiment, a method for the treatment of corneal ulceration, uveitis, or corneal transplant rejection in a subject is provided, comprising administering to the subject the composition for the treatment of dry eye.

Still another embodiment provides a method wherein the composition is applied topically to the eye of the subject.

### DETAILED DESCRIPTION OF THE INVENTION

Dry eye disease or syndrome is a multifactorial disorder of the tears and ocular surface, and is characterized by symptoms of dryness and irritation. It has been shown that inflammation is an important component in the development and propagation of dry eye (Stevenson et al., Arch. Ophthalmol., 2012, 130(1), 90-100; Rashid et al., Arch. Ophthalmol, 2008, 126(2), 219-225).

It has also been widely established that polyunsaturated fatty acids (herein referred to as PUFA(s) or FA(s)), particularly of the omega-3, and omega-6 series, are anti-inflammatory agents; they have shown beneficial effects in conditions associated with inflammation such as arthritis and ulcerative colitis. Preliminary studies have shown that alpha-linolenic acid (ALA) omega-3 FA can reverse the underlying inflammation developed with dry eye (Rashid et al., Arch. Ophthalmol, 2008, 126(2), 219-225, which is incorporated herein by reference in its entirety).

In one aspect, provided herein is a compostion for the treatment of dry eye comprising a pharmaceutically acceptable carrier and a pharmaceutically acceptable amount of a fatty acid salt of Formula I:

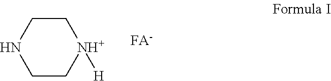

a fatty acid salt of Formula II:

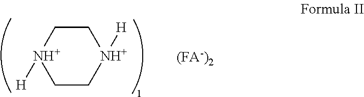

a fatty acid salt of Formula III:

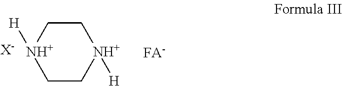

a fatty acid salt of Formula IV:

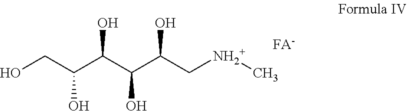

a fatty acid salt of Formula V:

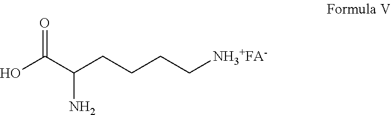

a fatty acid salt of Formula VI:

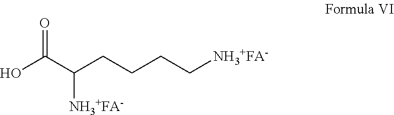

a fatty acid salt of Formula VII:

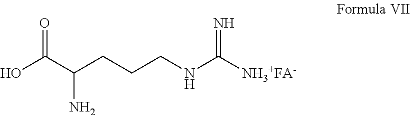

or a fatty acid salt of Formula VIII:

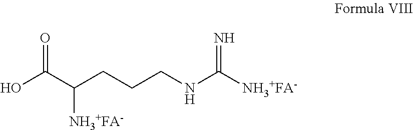

wherein FA⁻ is an anion of an omega-3 or omega-6 polyunsaturated fatty acid, and X⁻ is an anion of a pharmaceutically acceptable acid compound, or a combination thereof.

X⁻ is a pharmaceutically acceptable counter anion. The pharmaceutically acceptable counter anion can be derived from acid compounds listed in Table 1, pp 406-407, Handbook of Pharmaceutical Salts, P. Heinrich Stahl Camille G. Wermuth (Eds.). In an embodiment, the pharmaceutically acceptable counter anion is selected from mineral acids, such as hydrochloric acid, hydrobromic acid, and phosphoric acid. In another embodiment, the pharmaceutically acceptable counter anion is selected from carboxylic acids, poly-carboxylic acids, and poly-hydroxy carboxylic acids, such as acetic acid, propionic acid, succinic acid, maleic acid, malic acid, tartaric acid, lactic acid, citric acid, and benzoic acid. In another embodiment, the pharmaceutically acceptable counter anion is selected from sulfonic acids and hydroxyl-sulfonic acids, including, but not limited to, methanesulfonic acid, isethionic acid, ethanesulfonic acid, 2-hydroxy-ethanesulfonic acid, and benzenesulfonic acid. In another embodiment, the pharmaceutically acceptable counter anion is selected from amino acids, including, but not limited to, glycine, alanine, lysine, arginine, aspartic acid, or glutamic acid. In another embodiment, X⁻ is an omega-3 polyunsaturated acid, such as eicosapentaenoic acid or docosahexaenoic acid.

In an embodiment, X⁻ is mandelic acid.

In some embodiments provided herein are the following fatty acid salts: the hydrochloride salt of Formula III, the hydrobromide salt of Formula III, the phosphate salt of Formula III, and the sulfate salt of Formula III.

The present invention also relates to fatty acid salts of the Formula III wherein X⁻ is a pharmaceutically acceptable counter anion derived from naturally occurring amino acids. Examples of the amino acids include, but are not limited to, glycine, alanine, lysine, and glutamic acid.

In some embodiments, the fatty acid salt is a compound of Formula I, Formula II, or Formula III.

In some embodiments, FA⁻ is an anion of an omega-3 fatty acid, and the omega-3 fatty acid is selected from the group consisting of alpha-linolenic acid, stearidonic acid, eicosapentaenoic acid, and docosahexaenoic acid.

In further embodiments, FA⁻ is an anion of an omega-3 fatty acid, and the omega-3 fatty acid is eicosapentaenoic acid or docosahexaenoic acid.

In yet further embodiments, FA⁻ is an anion of an omega-6 fatty acid, and the omega-6 fatty acid is selected from the group consisting of linoleic acid, gammalinolenic acid, dihomogammalinoleic acid, and arachidonic acid.

The fatty acid salts of the invention, e.g., compounds of Formula I, II, III, IV, V, VI, VII, or VIII also include isomers and enantiomers wherever it is applicable.

Also provided herein are solvates (e.g., hydrates) of the fatty acid salts of the invention, e.g., compounds of Formula I, II, III, IV, V, VI, VII, or VIII. As used herein, the term “solvate” refers to any form of the fatty acid salts of the invention that are bound by a non-covalent bond to another molecule (such as a polar solvent). Such solvates are typically crystalline solids having a substantially fixed molar ratio of solute and solvent. When the solvent is water, the solvate formed is a hydrate. Example hydrates include hemihydrates, monohydrates, dihydrates, etc.

### Methods of Treatment

In one aspect, provided herein is a method for the treatment of dry eye in a subject in need thereof, comprising administering to the subject the composition of the invention, e.g. a composition comprising a pharmaceutically acceptable carrier and a pharmaceutically effective amount of a fatty acid salt of Formula I, II, III, IV, V, VI, VII, or VIII.

Dry eye is a multi-factorial disease of the tears and is generally characterized by symptoms of discomfort, visual disturbance, and tear film instability with potential damage to the ocular surface (exposed eye ball, sclera, conjunctival sac, cornea). Dry eye is associated with increased osmolality of the tear film and inflammation of the ocular surface due to improper secretion or production of various secretory glands in the eye lids including lacrimal glands.

Examples of dry eye include, but are not limited to alacrima, xerophthalmia, Sjogren's syndrome associated dry eye, non-Sjogren's syndrome associated dry eye, dry keratoconjunctivitis, ocular pemphigoid, dry eye after ophthalmic operation, and dry eye accompanied with conjunctivitis.

Accordingly, in one embodiment, provided herein is a method of treating alacrima, xerophthalmia, Sjogren's syndrome associated dry eye, non-Sjogren's syndrome associated dry eye, dry keratoconjunctivitis, ocular pemphigoid, dry eye after ophthalmic operation, or dry eye accompanied with conjunctivitis in a subject in need thereof, comprising administering to the subject a composition of the invention, e.g. a composition comprising a pharmaceutically acceptable carrier and a pharmaceutically effective amount of a fatty acid salt of Formula I, II, III, IV, V, VI, VII, or VIII.

In another embodiment, provided herein is a method of treating aqueous tear-deficient dry eye or evaporative dry eye in a subject in need thereof, comprising administering to the subject a composition of the invention, e.g. a composition comprising a pharmaceutically acceptable carrier and a pharmaceutically effective amount of a fatty acid salt of Formula I, II, III, IV, V, VI, VII, or VIII.

In another aspect, provided herein is a method for increasing tear production in a subject in need thereof, comprising administering to the subject a composition of the invention, e.g., a composition comprising a pharmaceutically acceptable carrier and a pharmaceutically effective amount of fatty acid salt of Formula I, II, III, IV, V, VI, VII, or VIII.

In yet another aspect, provided herein is a method for the treatment of corneal ulceration, uveitis, or corneal transplant rejection in a subject, comprising administering to the subject the composition of the invention, e.g., a composition comprising a pharmaceutically acceptable carrier and a pharmaceutically effective amount of a fatty acid salt of Formula I, II, III, IV, V, VI, VII, or VIII.

In an embodiment of the methods provided herein, the composition of the invention is applied topically to the eye of the subject.

The term “treat,” “treated,” “treating” or “treatment” includes the diminishment or alleviation of at least one symptom associated or caused by the state, disorder or disease being treated. In certain embodiments, the treatment comprises the induction of a disease, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

As used herein, the term “dry eye” refers to inadequate tear production and/or abnormal tear composition. Causes of dry eye disease as defined herein include but are not limited to the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjogren's syndrome and autoimmune diseases associated with Sjogren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Further, the term “dry eye” includes dry eye after anterior ophthalmic operation such as cataract operation and refractive surgery and that accompanied with allergic conjunctivitis.

Dry eye symptoms as defined herein may also be provoked by other circumstances, including, but not limited to, the following: prolonged visual tasking; working on a computer; being in a dry environment; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

As used herein, the term “a subject in need of the treatment of dry eye” or “a subject in need thereof' includes both a patient who has the dry eye condition and a patient who is suspected to be suffered from dry eye. The administration route is not specifically limited and topical ocular administration is preferable.

The term “subject” is intended to include organisms, e.g., prokaryotes and eukaryotes, which are capable of suffering from or afflicted with a disease, disorder or condition. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from dry eye.

The language “effective amount,” “pharmaceutically effective amount” or “pharmaceutically acceptable amount” of the compound is that amount necessary or sufficient to treat or prevent a disorder, e.g., prevent the various morphological and somatic symptoms of dry eye.

### Compositions for Topical Administration to the Eye

In another embodiment, the composition for the treatment of dry eye comprising a pharmaceutically acceptable carrier and a pharmaceutically effective amount of a fatty acid salt of Formula I, II, III, IV, V, VI, VII, or VIII, is formulated for topical administration to the eye of the subject.

In a further embodiment, the composition further comprises one or more of glycerin, hypromellose, propylene glycol or polyethylene glycol.

In yet a further embodiment, the composition further comprises one or more of polysorbate 80, carbomer copolymer type A, purified water, sodium hydroxide, ascorbic acid, benzalkonium chloride, boric acid, dextrose, disodium phosphate, glycine, magnesium chloride, potassium chloride, sodium borate, sodium chloride, sodium citrate, sodium lactate, edetate disodium, hydrochloric acid, sodium hydroxide, aminomethylpropanol, hydroxypropyl guar, polyquaternium-1, or sorbitol.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Formulations of the present invention include those suitable for topical administration to the eye. Preferably, the compositions administered according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

The compositions administered according to the present invention may also include various other ingredients, including but not limited to surfactants, tonicity agents, buffers, preservatives, co-solvents and viscosity building agents. Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm). An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6-7.5.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more of the fatty acid salts of the invention.

Examples or artificial tears compositions useful as artificial tears carriers include, but are not limited to, commercial products, such as Tears Naturale™, Tears Naturale II™, Tears Naturale Free™, and Bion Tears™. (Alcon Laboratories, Inc., Fort Worth, Tex.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Gionek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as polyvinyl alcohol, polyvinylpyrrolidone, povidone and carbomers, such as carbomer 934P, carbomer 941, carbomer 940, carbomer 974P. Other compounds may also be added to the ophthalmic compositions of the present invention to increase the viscosity of the carrier.

Examples of viscosity enhancing agents include, but are not limited to polysaccharides, such as hyaluronic acid and its salts, chondroitin sulfate and its salts, dextrans, various polymers of the cellulose family; vinyl polymers; and acrylic acid polymers. In general, the phospholipid carrier or artificial tears carrier compositions will exhibit a viscosity of 1 to 400 centipoises (“cps”). Topical ophthalmic products are typically packaged in multidose form.

Preservatives may be required to prevent microbial contamination during use. Suitable preservatives include benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Other wetting agents, emulsifiers and lubricants, such as sodium lauryl sulfate and magnesium stearate, as well as coloring agents, release agents, coating agents, and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

A contact lens may optionally be used to allow for extravasation of vasoactive substance over a more prolonged time period. Vasoactive substances such as Thrombin and Thromboxane A may further induce an increase in tear volume via venular vasoconstriction and increased perfusion through lacrimal, accessory lacrimal and surface microvessels. Increased paracellular endothelial openings that increase capillary permeability can further enhance this benefit.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers.

### Dosage

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, one to two drops of such compositions will be administered from once to many times per day.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

### Kits

Advantageously, the present invention also provides kits for use by a consumer for treating disease. The kits comprise a) a pharmaceutical composition comprising a compound of the invention and a pharmaceutically acceptable carrier, vehicle or diluent; and, optionally, b) instructions describing a method of using the pharmaceutical composition for treating the specific disease.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material.

During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

One embodiment of the present invention relates to a kit comprising a unit dosage comprising a compound of the invention with instructions on how to use the kit and with provision for at least one container for holding the unit dosage form.

### Methods of Making

The salts of the invention can be prepared using any number of synthesis techniques known to the skilled artisan.

An example for the synthesis of the mono salt of piperazine with eicosapentaenoic acid (EPA) (an example of the fatty acid salt of Formula I) can be prepared as set forth below.

One equivalent of piperazine may be dissolved in an appropriate reaction inert solvent. The solvent may be polar such as water. As used herein, the expression “reaction inert solvent” refers to a solvent or a mixture of solvents that does not interact with starting materials, reagents, intermediates or products in a manner that adversely affects the yield of the desired product. Preferred solvents include methanol, ethanol, n-propanol, isopropanol, acetone, ethyl methyl ketone, diethyl ketone, methyl isobutylketone and acetonitrile. To this solution may be added a solution of one equivalent of EPA. Both piperazine and EPA are commercially available. The reaction mixture can be stirred at about ambient temperature to about the reflux temperature of the solvent being used for about two hours to about six hours. The mono salt of piperazine with EPA can be isolated from the mixture by methods well known to those skilled in the art, including according to the method of U.S. Pat. No. 7,973,073.

The di-salt of piperazine with EPA (an example of the fatty acid salt of Formula II) can be prepared according to the above procedure, but by using two equivalents instead of one equivalent of EPA.

The mono salt of piperazine with docosahexaenoic acid (DHA) (an example of the fatty acid salt of Formula I) can be prepared as set forth below.

One equivalent of piperazine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of DHA. Both piperazine and DHA are commercially available. The reaction mixture can be stirred at about ambient temperature to about the reflux temperature of the solvent being used for about two hours to about six hours. The mono salt of piperazine with DHA can be isolated from the mixture by methods well known to those skilled in the art, including according to the method of U.S. Pat. No. 7,973,073.

The di-salt of piperazine with DHA (an example of the fatty acid salt of Formula II) can be prepared according to the above procedure, but by using two equivalents instead of one equivalent of DHA.

The fatty acid salt of Formula III can be prepared by adding a solution of one equivalent of compound XH to a solution of one equivalent of the compound of the Formula I. Suitable solvents include methanol, ethanol, n-propanol, isopropanol, acetone, ethyl methyl ketone, diethyl ketone, methyl isobutylketone and acetonitrile.

The meglumine salt of EPA (an example of the fatty acid salt of Formula IV) can be prepared as set forth below.

One equivalent of meglumine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of EPA. Both meglumine and EPA are commercially available. The reaction mixture can be stirred at about ambient temperature to about the reflux temperature of the solvent being used for about two hours to about six hours. The meglumine salt of EPA can be isolated from the mixture by methods well known to those skilled in the art, including according to the method of U.S. Pat. No. 7,973,073.

The meglumine salt of DHA (an example of the fatty acid salt of Formula IV) can be prepared as set forth below.

One equivalent of meglumine may be dissolved in an appropriate reaction inert solvent. The solvent may be polar such as water. Preferred reaction inert solvents include methanol, ethanol, n-propanol, isopropanol, acetone, ethyl methyl ketone, diethyl ketone, methyl isobutylketone and acetonitrile. To this solution may be added a solution of one equivalent of DHA. Both meglumine and DHA are commercially available. The reaction mixture can be stirred at about ambient temperature to about the reflux temperature of the solvent being used for about two hours to about six hours. The meglumine salt of DHA can be isolated from the mixture by methods well known to those skilled in the art, including according to the method of U.S. Pat. No. 7,973,073.

### Methods of Testing

The compositions described herein can be tested using the rabbit forced eyelid opening dry eye model as described below.

JW/CSK rabbits can be used. The animals are anesthetized by subcutaneous injection of urethane (2 g/kg, 8 mL/kg). The eyelids of the rabbits are forced to open by use of an eye speculum and the animals are held for 3 hours in the face-down position using a rabbit holder. After the forced eyelid opening is finished, the animals are sacrificed by intravenous administration of an excessive amount of 5% pentobarbital and the eyeballs were removed. 50 μl of 1% methylene blue solution is dropped on the surface of the cornea of the removed eyeball and stands for one minute. Then, the eyeball is put into a container containing 15 ml of physiological saline to wash the methylene blue. The cornea is removed with a razor and is soaked in acetone/sodium sulfate solution (100% acetone: 9% aqueous Na₂SO₄=7:3) for one night so that methylene blue can be extracted from the cornea into the solution. The amount of the methylene blue in the extract solution can be determined by measuring optical density at 660 nm using Multskan® Spectrum Thermo. Average absorbance±standard error can be determined and the corneal injury is evaluated by the average absorbance (i.e. methylene blue absorbance).

The coreneal injury inhibiting ratio of each test group will be evaluated according to the following criteria:

Average methylene blue absorbance of the untreated group is considered 100% inhibition of corneal injury.

Average methylene blue absorbance of the group receiving the forced eyelid opening and administered with physiological saline is considered 0% inhibition of the corneal injury.

The corneal injury inhibiting ratio of the test group can be calculated as follows:

$$Corneal injury inhibiting ratio ( \% ) = \frac{( Group with forced eyelid opening plus saline ) - ( test group )}{\begin{matrix}( Group with forced eyelid opening plus saline ) - \\ ( group with forced eyelid opening plus no treatment )\end{matrix}} .$$

The test group is the group subjected to the method described above, with administration of the composition of the invention immediately after starting the forced eyelid opening.

### EXEMPLIFICATION

### Example 1

### Preparation of Piperazine Eicosapentaenoate R-(−)-Mandelate

A solution of piperazine (0.450 g, 5.22 mmol) in acetonitrile (30 mL, 600 mmol) was treated with a solution of (5Z,8Z,11Z,14Z,17Z)-icosa-5,8,11,14,17-pentaenoic acid (1.90 g, 6.27 mmol) in acetonitrile (30 mL, 600 mmol). The solution was stirred for 10 minutes, and then cooled at 0° C. Upon cooling a white precipitate formed. The suspension was treated drop wise over 30 minutes with a solution of R-(−)-mandelic acid (0.795 g, 5.22 mmol) in acetonitrile (24 mL, 460 mmol) and the mixture was stirred an additional 2.5 h at 0-5° C. The reaction mixture was filtered under nitrogen and the solid was washed with cold acetonitrile. The solid was quickly transferred to a round bottom flask and placed under high vacuum overnight. Yield was 2.40 g. The ¹H NMR spectrum, the ¹³C NMR spectrum and elemental analysis indicate the material is piperazine eicosapentaenoate R-(−)-mandelate.

Anal Calcd for C32H48N2O5 plus 0.69% H2O: C, 70.59; H, 8.96; N, 5.14. Found: C, 70.27; H, 8.84; N, 5.12.

¹H NMR (300 MHz, MeOD) d ppm 7.47 (m, 2H), 7.27 (m, 3H), 5.36 (m, 10H), 3.05 (s, 8H), 2.85 (m, 8H), 2.28 (t, J=7.45 Hz, 2H), 2.10 (m, 4H), 1.67 (m, 2H), 0.97 (t, J=7.54 Hz, 3H)

¹³C NMR (101 MHz, MeOD) d ppm 179.42, 178.49, 143.34, 132.93, 130.37, 129.88, 129.59, 129.42, 129.31, 129.27, 129.20, 129.08,128.57, 128.33, 128.07, 76.10, 44.22, 35.18, 27.83, 26.70, 26.58, 26.42, 21.65, 14.81

### Example 2

### Bis[(5Z,8Z,11Z,14Z,17Z)-icosa-5,8,11,14,17-pentaenoatel Piperazine Salt (EPA2-Pip)

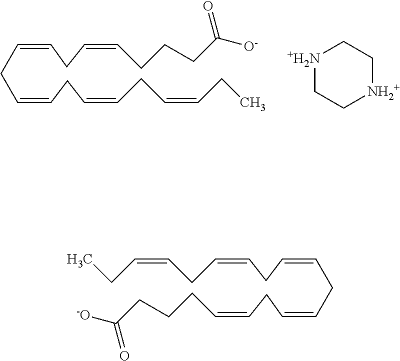

A stirred solution of piperazine (1.28 g, 14.9 mmol in acetonitrile (30 mL, 600 mmol) is treated dropwise with a solution of (5Z,8Z,11Z,14Z,17Z)-icosa-5,8,11,14,17-pentaenoic acid (9.00 g, 29.8 mmol) in acetonitrile (73 mL, 1400 mmol) under N₂. After 30 minutes, the mixture is stored in the refrigerator overnight. The solid was collected by filtration and dried under hi-vac at RT overnight with P₂O₅. Yield=8.2 g of bis[(5Z,8Z,11Z,14Z,17Z)-icosa-5,8,11,14,17-pentaenoate]piperazine salt as a pink solid.

¹¹H NMR (300 MHz, MeOD) δ 0.99 (t, 6H) 1.68 (t, 4H) 2.04-2.18 (m, 3H) 2.11 (d, 5H) 2.25 (t, 4H) 2.85 (m, 17H) 3.07 (s, 9H) 5.27-5.45 (m, 20H); MS (ESI+) for C₂₀H₃₀O₂ m/z 303 (M+H)⁺; Anal Calcd for C₄₄H₇₀N₂O₄: C, 76.48; H, 10.21; N, 4.05. Found: C, 76.54; H, 10.09; N, 4.04.; MP=61-64° C.

### Example 3

### Ethane-1,2-diaminium di[(5Z,8Z,11Z,14Z,17Z)-icosa-5,8,11,14,17-pentaenoate] (EPA2-EDA)

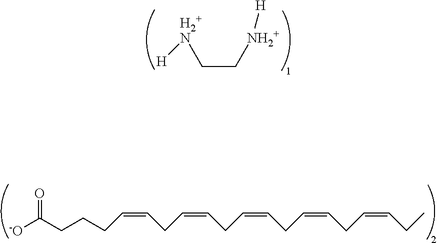

A mixture of (5Z,8Z,11Z,14Z,17Z)-icosa-5,8,11,14,17-pentaenoic acid (10.50 g, 34.72 mmol) and ethylenediamine (1.10 mL, 16.5 mmol) in acetonitrile (215.8 mL, 4133 mmol) is stirred with ice bath cooling for 2 hrs and then stored in the refrigerator overnight. The solid is collected by filtration and dried under hi-vac at RT over P₂O₅ for 12 hrs. Yield=7.9 g of ethane-1,2-diaminium di[(5Z,8Z,11Z,14Z,17Z)-icosa-5,8,11,14,17-pentaenoatel as a brown solid. ¹H NMR (300 MHz, MeOD) δ 0.92 (t., 6H) 1.65 (m, 4H) 2.16 (m, 12H) 2.89 (m., 16H) 2.99 (s, 4H) 4.88 (s, 6H) 5.37 (br. s., 20H); MS (ESI+) for C₂₀H₃₀O₂ m/z 303 (M+H)⁺; Anal Calcd for C₄₂H₆₈N₂O₄: C, 75.86; H, 10.31; N, 4.21. Found: C, 75.70; H, 10.25; N, 4.07. MP=30° C.

### Example 4

### (Benzoylamino)acetate (5Z,8Z,11Z,14Z,17Z)-icosa-5,8,11,14,17-pentaenoate Piperazine Salt (EPA Piperazine Hippurate Salt)

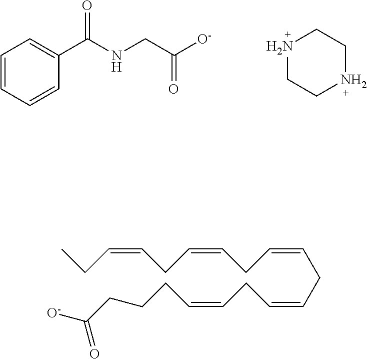 [SMILES: O=C([O-])CNC(=O)c1ccccc1 ; C1CNCCN1 ; CC/C=C\C/C=C\C/C=C\C/C=C\C/C=C\CCCC(=O)[O-]]

A solution of (5Z,8Z,11Z,14Z,17Z)-icosa-5,8,11,14,17-pentaenoic acid (7.00 g, 23.1 mmol) in 100 mL acetonitrile is added to a solution of piperazine (1.661 g, 19.29 mmol) in 90 mL acetonitrile and the mixture is stirred for 30 min. The mixture is cooled in an ice bath and a solution of N-benzoylglycine (3.456 g, 19.29 mmol) in 80 mL of acetonitrile is added drop wise. The mixture is stirred with ice bath cooling for 6 hours, then stored in the refrigerator overnight. The solid is collected by filtration and rinsed with 3×20 ml of ice cold CH₃CN. The resulting solid is dried at RT under hi-vac with P₂O₅ overnight. Yield=9.1 g of (benzoylamino) acetate (5Z,8Z,11Z,14Z,17Z)-icosa-5,8,11,14,17-pentaenoate piperazine salt as a pink solid. ¹H NMR (300 MHz, MeOD) d ppm 0.98 (t, J=7.54 Hz, 3H) 1.51-1.79 (m, 2H) 1.94-2.22 (m, 4H) 2.28 (t, J=7.45 Hz, 2H) 2.66-2.95 (m, 8H) 5.12-5.56 (m, 8H) 7.28-7.63 (m, 3H) 7.75-7.98 (m, 2H). Anal Calcd for C₃₃H₄₉N₃O₅ plus 0.28% H₂O: C, 69.62; H, 8.71; N, 7.38. Found: C, 69.26; H, 8.35; N, 7.58.

### Example 5

### Ethane-1,2-diaminium (5Z,8Z,11Z,14Z,17Z)-icosa-5,8,11,14,17-pentaenoate methanesulfonate

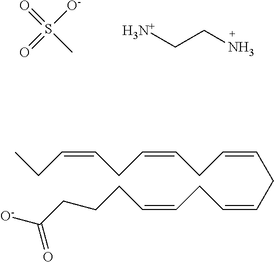

A mixture of ethylenediamine (0.884 mL, 13.2 mmol) and (5Z,8Z,11Z,14Z,17Z)-icosa-5,8,11,14,17-pentaenoic acid (4.800 g, 15.87 mmol; Supplier=Thetis) in acetonitrile (138.1 mL, 2645 mmol) is stirred at RT for ½ hr. Methanesulfonic acid (0.858 mL, 13.2 mmol) is added and the mixture is stirred with ice bath cooling for 1 hr, then stored in the refrigerator overnight. The solid is collected by filtration and washed with ice cold CH₃CN. Yield=4.5 g of ethane-1,2-diaminium (5Z,8Z,11Z,14Z,17Z)-icosa-5,8,11,14,17-pentaenoate methanesulfonate as a tacky solid. The material is triturated with ice cold CH₃CN (3×20 ml) and dried under hi-vac with P₂O₅ at RT overnight. Yield=3.4 g of ethane-1,2-diaminium 5Z,8Z,11Z,14Z,17Z)-icosa-5,8,11,14,17-pentaenoatemethane sulfonate as a tan solid. ¹H NMR (300 MHz, DMSO-d₆) d ppm 0.92 (t, J=7.54 Hz, 3H) 1.44-1.63 (m, 2H) 1.95-2.10 (m, 2H) 2.16 (t, J=7.40 Hz, 2H) 2.34 (s, 3H) 2.69-2.91 (m, 12H) 5.20-5.46 (m, 10H) 6.60 (br. s., 6H). Anal Calcd for C₂₃H₄₂N₂O₅S plus 0.60% H₂O: C, 59.87; H, 9.24; N, 6.07. Found: C, 59.48; H, 9.13; N, 6.24.

### Example 6

### 1-deoxy-1-(methylamino)-D-glucitol (5Z,8Z,11Z,14Z,17Z)-icosa-5,8,11,14,17-pentaenoate

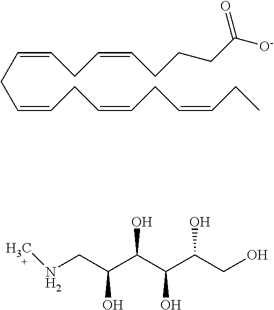 [SMILES: CC/C=C\C/C=C\C/C=C\C/C=C\C/C=C\CCCC(=O)[O-] ; CNC[C@H](O)[C@@H](O)[C@H](O)[C@H](O)CO]

A suspension of 1-deoxy-1-(methylamino)-D-glucitol (0.200 g, 1.02 mmol) in 6.0 mL of methanol was treated drop wise with a solution of (5Z,8Z,11Z,14Z,17Z)-icosa-5,8,11,14,17-pentaenoic acid (0.325 g, 1.08 mmol) in 7.0 mL tetrahydrofuran and the mixture was stirred at RT for 30 minutes while being protect from light. The slurry became homogeneous upon addition of the EPA solution. The solution was concentrated to pink oil that was placed on high vac overnight to yield a pink foam. ¹H NMR (300 MHz, MeOD) δ ppm 5.36 (m, 10H), 4.04 (dt, J=6.94, 4.96 Hz, 1H), 3.80 (m, 2H), 3.68 (m, 3H), 3.14 (m, 2H), 2.84 (m, 8H), 2.68 (s, 3H), 2.19 (m, 2H), 2.10 (m, 4H), 1.66 (m, 2H), 0.97 (t, J=7.50 Hz, 3H).

### Example 7

### Pharmacokinetics of Piperazine di-eicosapentaenoate

Oral pharmacokinetic parameters of piperazine di-eicosapentaenoate, prepared by the procedure described in Example 2, were determined in Sprague-Dawley rats. Piperazine di-eicosapentaenoate was administered by oral gavage as an aqueous solution in 0.5% carboxymethyl cellulose to 6 Sprague-Dawley rats, 3 males and 3 females. Rats were dosed at 40 mg/kg. Blood samples were obtained from each rat by jugular vein catheter. Samples were collected at 0.25, 0.5, 1, 2, 4, 8, 12, and 24 hours post dose. Blood samples were centrifuged to separate red blood cells and the resulting plasma samples were analyzed for eicosapentaenoic acid. Calculated pharmacokinetic parameters shown below in Table 1 are mean values from 6 rats.

TABLE 1
Rat Oral Pharmacokinetic Parameters for Piperazine
di-eicosapentaenoate

| Analyte | EPA |
|---|---|
| Cₘₐₓ (μg/mL) | 28.22 |
| Tₘₐₓ (h) | 1.0 |
| AUC (0-24) | 134.85 |
| (μg * h/mL) | |

### Example 8

### Pharmacokinetics of Ethylene diamine di-eicosapentaenoate

Oral pharmacokinetic parameters of ethylene diamine di-eicosapentaenoate, prepared by the procedure described in Example 3, were determined in Sprague-Dawley rats. Ethylene diamine di-eicosapentaenoate was administered by oral gavage as an aqueous solution in 0.5% carboxymethyl cellulose to 6 rats, 3 males and 3 females. Rats were dosed at 40 mg/kg. Blood samples were obtained from each rat by jugular vein catheter. Samples were collected at 0.25, 0.5, 1, 2, 4, 8, 12, and 24 hours post dose. Blood samples were centrifuged to separate red blood cells and the resulting plasma samples were analyzed for eicosapentaenoic acid. Calculated pharmacokinetic parameters shown below (Table 2) are mean values from 6 rats.

TABLE 2
Rat Oral Pharmacokinetic Parameters for Ethylene diamine
di-eicosapentaenoate

| Analyte | EPA |
|---|---|
| Cₘₐₓ (μg/mL) | 18.37 |
| Tₘₐₓ (h) | 2.0 |
| AUC (0-24) (μg * h/mL) | 275.29 |

1. A composition for the treatment of dry eye comprising a pharmaceutically acceptable carrier and a pharmaceutically effective amount of a fatty acid salt of Formula I: 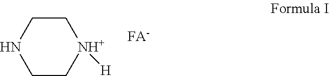
a fatty acid salt of Formula II: 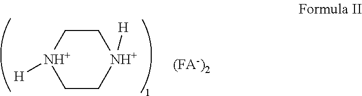
a fatty acid salt of Formula III: 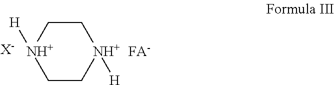
a fatty acid salt of Formula IV: 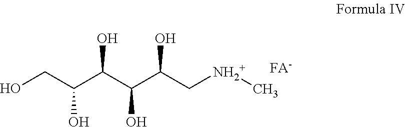
a fatty acid salt of Formula V: 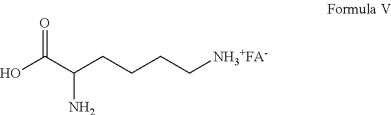
a fatty acid salt of Formula VI: 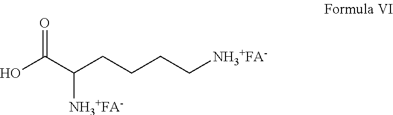
a fatty acid salt of Formula VII: 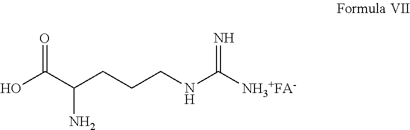
or a fatty acid salt of Formula VIII: 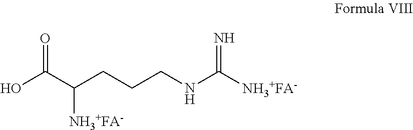
wherein FA⁻ is an anion of an omega-3 or omega-6 polyunsaturated fatty acid, and X⁻ is an anion of a pharmaceutically acceptable acid compound, or a combination thereof.

2. The composition of claim 1, wherein the fatty acid salt is a compound of Formula I, Formula II, or Formula III.

3. The composition of claim 1 wherein FA⁻ is an anion of an omega-3 fatty acid, and the omega-3 fatty acid is selected from the group consisting of alpha-linolenic acid, stearidonic acid, eicosapentaenoic acid, and docosahexaenoic acid.

4. The composition of claim 1, wherein FA⁻ is an anion of an omega-3 fatty acid, and the omega-3 fatty acid is eicosapentaenoic acid or docosahexaenoic acid.

5. The composition of claim 1, wherein FA⁻ is an anion of an omega-6 fatty acid, and the omega-6 fatty acid is selected from the group consisting of linoleic acid, gammalinolenic acid, dihomogammalinoleic acid, and arachidonic acid.

6. The composition of claim 1, formulated for topical administration to the eye of the subject in need thereof.

7. The composition of claim 6, further comprising one or more of glycerin, hypromellose, propylene glycol or polyethylene glycol.

8. The composition of claim 6, further comprising one or more of polysorbate 80, carbomer copolymer type A, purified water, sodium hydroxide, ascorbic acid, benzalkonium chloride, boric acid, dextrose, disodium phosphate, glycine, magnesium chloride, potassium chloride, sodium borate, sodium chloride, sodium citrate, sodium lactate, edetate disodium, hydrochloric acid, sodium hydroxide, aminomethylpropanol, hydroxypropyl guar, polyquaternium-1, or sorbitol.

9. A method for the treatment of dry eye in a subject in need thereof, comprising administering to the subject the composition of claim 1.

10. The method of claim 9, wherein dry eye is selected from the group consisting of alacrima, xerophthalmia, Sjogren's syndrome associated dry eye, non-Sjogren's syndrome associated dry eye, dry keratoconjunctivitis, ocular pemphigoid, dry eye after ophthalmic operation, and dry eye accompanied with conjunctivitis.

11. The method of claim 9, wherein dry eye is aqueous tear-deficient dry eye or evaporative dry eye.

12. A method for increasing tear production in a subject comprising administering to the subject the composition of claim 1.

13. A method for the treatment of corneal ulceration, uveitis, or corneal transplant rejection in a subject, comprising administering to the subject the composition of claim 1.

14. The method of claim 9, wherein the composition is applied topically to the eye of the subject.
